(19) United States
(12) Patent Application Publication
(10) Pub. No.: US 2025/0304517 A1
(43) Pub. Date: Oct. 2, 2025

(54) STERIOISOMER-SPECIFIC CANNABIS FORMULATIONS AND ANALYSIS

(71) Applicant: Scientific Holdings, LLC, Commerce, CA (US)

(72) Inventors: Jeffrey Charles Raber, Commerce, CA (US); Bradley J. Douglass, Commerce, CA (US); Sytze Elzinga, Commerce, CA (US); Braden Doane, Commerce, CA (US)

(73) Assignee: Scientific Holdings, LLC, Commerce, CA (US)

(21) Appl. No.: 19/236,844

(22) Filed: Jun. 12, 2025

Related U.S. Application Data
(63) Continuation of application No. 17/139,866, filed on Dec. 31, 2020.

(51) Int. Cl. C07C 33/02; C07C 13/20; C07C 13/605

(52) U.S. Cl. CPC C07C 33/02; C07C 13/20; C07C 13/605

(57) ABSTRACT

A synthetic chiral composition comprising (i) pinene isomers; (ii) linalool isomers; and (iii) a terpene or terpenoid, and formulations comprising the synthetic chiral composition, in addition to methods for preparing, creating, populating, and querying databases pertaining to, and kits comprising, the synthetic chiral composition are disclosed herein. The composition further includes one or more modifiers. The composition includes organoleptic properties (e.g. aroma) of a plant cultivar (e.g. Cannabis).

This patent application is a continuation of, and claims priority to, U.S. patent application Ser. No. 17/139,866 filed on Dec. 31, 2020, which is incorporated by reference herein in its entirety.

### TECHNICAL FIELD

The present disclosure relates to an imitation composition prepared based on a chemical profile of plants, and more particularly, to a synthetic chiral composition prepared based on a stereospecific terpene profile that possesses improved organoleptic, physiological, and toxicological properties of cannabis varieties.

### BACKGROUND

Cannabis is a genus of flowering plants in the family Cannabaceae and cannabis cultivars are either pure or hybrid varieties of the plant genus Cannabis, which has long been used for drug (cannabinoids) and industrial (hemp) purposes. Cannabis plant material has been reported to contain beneficial compounds such as cannabinoids, terpenes, and flavonoids. Terpenes are an important subset of cannabis constituents and-unlike cannabinoids and flavonoids-impact the taste and aroma of cannabis.

In recent years, an accurate view of the chemical profile of cannabis cultivars or varieties (cf. strain) is of interest for many reasons. Generally, standard analytical chemistry techniques, such as gas chromatography (GC) and high-performance liquid chromatography (HPLC), are used to chemically-profile plants and classify cannabis cultivars by chemical composition (chemotype). This is sometimes referred to colloquially as a cultivar “fingerprint.” GC and HPLC data are applied to discriminate cannabis varieties and are used as a basis for creating synthetic compositions that possess cultivar-specific properties (e.g. aroma, taste, effects). That is, an essential set of molecules (e.g. terpenes) present amongst the chemical-profile of cannabis cultivars is thereby selected to create the synthetic composition/formulation that maintains desirable characteristics while eliminating undesirable characteristics. However, the composition may have fewer overall components, including some toxicologically suspect molecules, especially when heated to high temperatures, which have been reported in cannabis plants and are sometimes present in natural cannabis extracts, such as ledol, pulegone, or myrcene. Hence, further discrimination amongst cannabis cultivars is needed to create desirable compositions and to understand which chemically distinct molecules are responsible for the overall additive physiological effects of the composition.

It is well-known that terpenes with very different properties may differ by only the stereochemistry at a single carbon atom. One example of this is the enantiomeric pair of R-(−)-carvone and S-(+)-carvone: R-(−)-carvone possesses an odor characteristic of spearmint while S-(+)-carvone is redolent of caraway. Accordingly, there is a need to study the stereospecific profile, especially including enantiomers, using uncommon analytical techniques to further discriminate amongst plant cultivars and to determine the type and amount of each stereochemically-relevant terpene in the plant cultivars. The key is resolution and quantification of enantiomers. One suitable analytical technique for the analysis of enantiomers is Chiral Gas Chromatography (CGC). Enantiomeric analysis (i.e. analysis on stereoisomers of terpenoids) using Chiral Gas Chromatography (CGC) has been reported previously in the art to discriminate amongst some plant cultivars and/or to confirm their similarity. For example, cultivars of citrus fruit have been discriminated using CGC. However, there is no report for applying CGC to profile cannabis cultivars and for using CGC data to create stereospecific compositions that maintain cannabis cultivar-specific properties.

Accordingly, there remains a need for chiral/stereoisomer-specific imitation compositions and a method for preparing the chiral/stereoisomer-specific imitation composition that possesses improved organoleptic, physiological, and toxicological properties of cannabis varieties.

### SUMMARY

The present disclosure provides a synthetic chiral composition that mimics organoleptic properties of a plant cultivar.

In one embodiment, the organoleptic properties of the plant cultivar selected from the group consisting of Cannabis sativa (Cannabis sativa L ssp sativa), Humulus lupulus, Cannabis indica (Cannabis sativa L ssp indica), and Cannabis ruderalis, (Cannabis sativa L ssp ruderalis).

In one embodiment, the organoleptic properties of the plant cultivar are selected from the group consisting of aroma, flavor, and other physiological characteristics.

In one embodiment, said synthetic chiral composition comprises one or more pinene isomers.

In one embodiment, said synthetic chiral composition comprises one or more linalool isomers.

In one embodiment, said synthetic chiral composition comprises one or more terpenes and/or terpenoids.

In one embodiment, said synthetic chiral composition comprises at least one isomer of one or more terpenes and/or terpenoids.

In one embodiment, said synthetic chiral composition comprises one or more pinene isomers and one or more linalool isomers.

In one embodiment, said synthetic chiral composition comprises one or more pinene isomers and one or more terpenes and/or terpenoids in defined amounts and isomeric ratios when more than one isomer is present.

In one embodiment, said synthetic chiral composition comprises one or more linalool isomers and one or more terpenes and/or terpenoids in defined amounts and isomeric ratios when more than one isomer is present.

In one embodiment, said synthetic chiral composition comprises: (i) one or more pinene isomers; (ii) one or more linalool isomers; and (iii) one or more terpenes and/or terpenoids in defined amounts and isomeric ratios when more than one isomer is present.

In one embodiment, the one or more pinene isomers can be selected from the group consisting of α-pinene, β-pinene, and a combination thereof.

In one embodiment, the one or more pinene isomers can be selected from the group consisting of an α-pinene enantiomer, a β-pinene enantiomer, and a combination thereof.

In one embodiment, the one or more pinene isomers can be selected from the group consisting of 1R)-(+)-α-pinene, (1S)-(−)-α-pinene, (1R)-(+)-β-pinene, and (1S)-(−)-β-pinene.

In one embodiment, the one or more pinene isomers can be produced synthetically or purified from a natural source.

In one embodiment, said synthetic chiral composition comprises a ratio of (1R)-(+)-α-pinene to (1S)-(−)-α-pinene ranging from 100:1 to 1:100.

In one embodiment, said synthetic chiral composition comprises a ratio of (1R)-(+)-β-pinene to (1S)-(−)-β-pinene ranging from 100:1 to 1:100.

In one embodiment, said synthetic chiral composition comprises a total weight of the one or more pinene isomers in an amount of about 0.01% to about 25% by weight of the composition.

In one embodiment, the one or more linalool isomers comprises a linalool enantiomer.

In one embodiment, the one or more linalool isomers can be selected from the group consisting of (S)-(+)-linalool, (R)-(−)-linalool, and a combination thereof.

In one embodiment, said synthetic chiral composition comprises a ratio of (S)-(+)-linalool to (R)-(−)-linalool ranging from 100:1 to 1:100.

In one embodiment, said synthetic chiral composition with a total weight percentage of the one or more linalool isomers in an amount of about 0.01% to about 25%.

In one embodiment, the one or more terpenes and/or terpenoids are selected from the group consisting of alpha-bisabolol, beta-caryophyllene, borneol, camphene, camphor, delta-3-carene, caryophyllene oxide, alpha-cedrene, beta-eudesmol, fenchol, geraniol, guaiol, alpha-humulene, isoborneol, limonene, menthol, myrcene, nerol, cis-ocimene, trans-ocimene, alpha-phellandrene, sabinene, alpha-terpinene, alpha-terpineol, terpinolene, alpha-guaiene, elemene, farnesene, germacrene, guaia-1(10),11-diene, trans-2-pinanol, selina-3,7(11)-diene, eudesm-7(11)-en-4-ol, valencene, and a combination thereof.

In one embodiment, the one or more terpenes and/or terpenoids is one or more isomers of terpenes and/or terpenoids selected from the group consisting of alpha-bisabolol, beta-caryophyllene, borneol, camphene, camphor, delta-3-carene, caryophyllene oxide, alpha-cedrene, beta-eudesmol, fenchol, geraniol, guaiol, alpha-humulene, isoborneol, limonene, menthol, myrcene, nerol, cis-ocimene, trans-ocimene, alpha-phellandrene, sabinene, alpha-terpinene, alpha-terpineol, terpinolene, alpha-guaiene, elemene, farnesene, germacrene, guaia-1(10),11-diene, trans-2-pinanol, selina-3,7(11)-diene, eudesm-7(11)-en-4-ol, valencene, and a combination thereof.

In one embodiment, the one or more terpenes or terpenoids is/are present in an amount from about 0.01% to about 99% by weight of the composition.

In one embodiment, the one or more isomers of the one or more terpenes and/or terpenoids are purified from a natural source or are produced synthetically.

In one embodiment, said synthetic chiral composition further comprises one or more modifiers selected from the group consisting of a thiol, a sulfur compound, an ester, a ketone, an aldehyde, a cannabinoid, a flavonoid and a combination thereof.

In one embodiment, the cannabinoid is selected from the group consisting of Δ9-tetrahydrocannabinol (Δ9-THC), Δ8-tetrahydrocannabinol (Δ8-THC), tetrahydrocannabinol acid (THCA), tetrahydrocannabivarin (THCV), tetrahydrocannabivarin acid (THCVA), cannabidiol (CBD), cannabidiol acid (CBDA), cannabichromene (CBC), cannabidivarin (CBDV), cannabidivarinic acid (CBDVA), cannabigerol (CBG), cannabigerol acid (CBGA), cannabigerovarin (CBGV), cannabinol (CBN), cannabinovarin (CBNV), and combination thereof.

In one embodiment, said synthetic chiral composition comprises a total amount of one or more modifiers in an amount from about 0.01% to about 90% by weight of the composition.

In one embodiment, said synthetic chiral composition comprises a stereospecific terpene profile of the plant cultivar.

In one embodiment, the stereospecific terpene profile of the plant cultivar is obtained by analyzing the plant cultivar using a chiral gas chromatography (CGC).

The present disclosure also provides a kit comprising said synthetic chiral compositions.

The present disclosure also provides formulations comprising said synthetic chiral composition.

In one embodiment, said formulations comprise: (i) said synthetic chiral composition, and (ii) one or more additional terpenes or terpenoids.

In one embodiment, said formulations comprise: (i) said synthetic chiral composition, (ii) one or more additional terpenes or terpenoids, and (iii) a modifier.

In one embodiment, the one or more additional terpenes or terpenoids are selected from the group consisting of alpha-bisabolol, beta-caryophyllene, borneol, camphene, camphor, delta-3-carene, caryophyllene oxide, alpha-cedrene, beta-eudesmol, fenchol, geraniol, guaiol, alpha-humulene, isoborneol, limonene, menthol, myrcene, nerol, cis-ocimene, trans-ocimene, alpha-phellandrene, sabinene, alpha-terpinene, alpha-terpineol, terpinolene, alpha-guaiene, elemene, farnesene, germacrene, guaia-1(10),11-diene, trans-2-pinanol, selina-3,7(11)-diene, eudesm-7(11)-en-4-ol, valencene, and a combination thereof.

In one embodiment, the one or more additional terpenes and/or terpenoids is one or more isomers of terpenes and/or terpenoids selected from the group consisting of alpha-bisabolol, beta-caryophyllene, borneol, camphene, camphor, delta-3-carene, caryophyllene oxide, alpha-cedrene, beta-eudesmol, fenchol, geraniol, guaiol, alpha-humulene, isoborneol, limonene, menthol, myrcene, nerol, cis-ocimene, trans-ocimene, alpha-phellandrene, sabinene, alpha-terpinene, alpha-terpineol, terpinolene, alpha-guaiene, elemene, farnesene, germacrene, guaia-1(10),11-diene, trans-2-pinanol, selina-3,7(11)-diene, eudesm-7(11)-en-4-ol, valencene, and a combination thereof.

In one embodiment, the one or more additional terpenes or terpenoids in the formulation are purified from a natural source or are produced synthetically.

In one embodiment, the one or more additional terpenes or terpenoids in the formulation is myrcene.

In one embodiment, the one or more additional terpenes or terpenoids in the formulation is not myrcene.

In one embodiment, the one or more additional terpenes or terpenoids in the formulation is beta-caryophyllene.

In one embodiment, the one or more additional terpenes or terpenoids in the formulation is limonene.

In one embodiment, the one or more additional terpenes or terpenoids in the formulation is terpinolene.

In one embodiment, the one or more additional terpenes or terpenoids in the formulation is alpha-humulene.

In one embodiment, the one or more additional terpenes or terpenoids in the formulation is not (S)-(−)-limonene.

In one embodiment, the one or more additional terpenes or terpenoids in the formulation is neither ledol nor pulegone.

In one embodiment, the one or more additional terpenes or terpenoids in the formulation are beta-caryophyllene and limonene.

In one embodiment, the one or more additional terpenes or terpenoids in the formulation are beta-caryophyllene and terpinolene.

In one embodiment, said formulation comprises a total amount of about 2% (wt./vol.) to about 98% (wt./vol.) of said synthetic chiral composition.

In one embodiment, the modifier is present in a total amount of about 0.5% (wt./vol.) to 90% (wt./vol.) of the formulation.

In one embodiment, said formulation further comprises one or more inert compounds selected from the group consisting of excipients, viscosity-imparting agents, solvents, binders, lubricants, stabilizers, preservatives, and diluents.

In one embodiment, said formulation is formulated into a liquid, a slurry, an emulsion, a suspension, an aerosol, a gel, and the like, or coated on solid particulate matrix.

In one embodiment, said formulation is a perfume, an incense, a cosmetic, a moisturizer, an emollient, a toiletry, an edible substance, an inhalable substance, an e-cigarette liquid, a candle and the like.

The present disclosure also provides a kit comprising said formulations.

The present disclosure also provides methods for preparing a synthetic chiral composition.

In one embodiment, said method for preparing the composition comprises obtaining a sample from the plant cultivar and determining the stereospecific chemical profile of the sample using chiral gas chromatography (CGC).

In one embodiment, said method for preparing the composition comprises obtaining a sample from the plant cultivar and determining the stereospecific chemical profile of the sample using chiral liquid chromatography (CLC).

In one embodiment, said method for preparing the composition further comprises analyzing the stereospecific chemical profile of the sample to identify one or more compounds that have organoleptic properties of the plant cultivar.

In one embodiment, the one or more compounds identified is a terpene or terpenoid.

In one embodiment, the one or more compounds identified is selected from the group consisting of flavonol, flavonol glycoside, and flavonoid.

In one embodiment, said method for preparing the composition comprises quantifying the one or more compounds identified in the sample.

In one embodiment, said method for preparing the synthetic chiral composition comprises mixing one or more compounds that mimic the organoleptic or physiologically-differentiating properties of the one or more compounds identified in the sample.

In one embodiment, said method for preparing the synthetic chiral composition comprises preparing the synthetic chiral composition that mimics the organoleptic or physiologically-differentiating properties of the one or more compounds identified in the sample based on the one or more quantified compounds.

In one embodiment, the synthetic chiral composition prepared by said method comprises a synthetic form of the one or more compounds identified in the sample.

In one embodiment, the synthetic chiral composition prepared by said method comprises a natural form of the one or more compounds identified in the sample.

In one embodiment, the synthetic chiral composition prepared by said method comprises a synthetic and/or natural form of the one or more compounds identified in the sample.

The present disclosure also provides methods for generating a database of synthetic chiral compositions that mimics organoleptic and physiological properties of a plant cultivar.

In one embodiment, the database comprises a plurality of synthetic chiral compositions.

In one embodiment, said method for generating a database comprises obtaining a sample from the plant cultivar and determining the stereospecific chemical profile of the sample using chiral gas chromatography (CGC).

In one embodiment, said method for generating a database comprises obtaining a sample from the plant cultivar and determining the stereospecific chemical profile of the sample using chiral liquid chromatography (CLC).

In one embodiment, said method for generating a database comprises analyzing the stereospecific chemical profile of the sample to identify one or more compounds that have organoleptic properties of the plant cultivar.

In one embodiment, said one or more compounds identified is a terpene or terpenoid.

In one embodiment, said method for generating a database comprises quantifying the one or more terpenes identified in the sample.

In one embodiment, said method for generating a database comprises generating one or more synthetic chiral compositions using different combinations of the one or more quantified terpenes or terpenoids with the one or more quantified terpene.

In one embodiment, said method for generating a database comprises generating one or more synthetic chiral composition using different combinations of the one or more quantified terpenes or terpenoids with the one or more quantified terpene enantiomers.

In one embodiment, the one or more quantified terpene enantiomers are selected from the group consisting of an alpha-pinene enantiomer, a beta-pinene enantiomer, a linalool enantiomer or a combination thereof. In some embodiments, the one or more terpenes or terpenoids include alpha-bisabolol, borneol, camphene, camphor, delta-3-carene, caryophyllene oxide, alpha-cedrene, beta-eudesmol, fenchol, geraniol, guaiol, alpha-humulene, isoborneol, linalool, menthol, myrcene, nerol, cis-ocimene, trans-ocimene, alpha-phellandrene, alpha-pinene, beta-pinene, sabinene, alpha-terpinene, alpha-terpineol, terpinolene, alpha-guaiene, elemene, farnesene, germacrene, guaia-1(10),11-diene, trans-2-pinanol, selina-3,7(11)-diene, eudesm-7(11)-en-4-ol, valencene, a combination thereof.

In one embodiment, said method for generating a database comprises the one or more terpene enantiomers and terpenes or terpenoids identified in the samples with the quantity and ratio information.

In one embodiment, said one or more compounds identified is selected from the group consisting of flavonol, flavone, flavonol glycoside, and flavonoid.

In one embodiment, said method for generating a database comprises quantifying the one or more compounds selected from the group consisting of flavonols, flavones, flavonol glycosides, and flavonoids identified in the sample.

In one embodiment, said method for generating a database comprises generating one or more synthetic chiral compositions using different combinations of the one or more quantified flavonols, flavones, flavonol glycosides, or flavonoids with the one or more flavonols, flavonol glycosides, or flavonoids.

In one embodiment, said method for generating a database comprises generating one or more synthetic chiral composition using different combinations of the one or more quantified flavonols, flavones, flavonol glycosides, or flavonoids with the one or more flavonols, flavonol glycosides, or flavonoids enantiomers.

In one embodiment, the one or more quantified flavonols, flavones, flavonol glycosides, or flavonoids are selected from the group consisting of 3-hydroxyflavone, azaleatin, fisetin, galangin, gossypetin, kaempferide, kaempferol, isorhamnetin, morin, myricetin, natsudaidain, pachypodol, quercetin, rhamnazin, rhamnetin, apigenin, chrysin, luteolin, baicalin, baicalein, astragalin, azalein, cannaflavin A, cannaflavin B, cannaflavin C, hyperoside, isoquercetin, isovitexin, kaempferitrin, myrcitrin, quercitrin, robinin, spiraeoside, xanthohamnin, amurensin, icarlin, troxerutin, vitexin, and a combination thereof.

The present disclosure also provides methods for authenticating a plant cultivar, comprising determining amounts of isomers of one or more terpenes and/or terpenoids.

The present disclosure also provides methods for differentiating between two or more plant cultivars, comprising determining amounts of isomers of one or more terpenes and/or terpenoids in the two or more plant cultivars.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments described herein include all such modifications.

### BRIEF DESCRIPTION OF THE DRAWINGS

The embodiments described herein will be better understood from the following detailed description with reference to the drawings, in which:

FIG. 1 is a flow diagram that illustrates a method of preparing a synthetic chiral composition.

FIG. 2 is a flow diagram that illustrates a method of generating a database comprising a plurality of synthetic chiral compositions.

FIG. 3 is a graphical representation that shows a result of a chiral gas chromatographic analysis of strains of cannabis by enantiomer content, according to some embodiments described herein

FIG. 4 is a flow diagram that illustrates the method of analyzing an unknown sample and querying the knowledge base to establish similarity to known cultivars.

FIG. 5 is a flow diagram that illustrates a method of generating a knowledge base comprising a plurality of cultivar chemical profiles.

### DETAILED DESCRIPTION

As mentioned above, there remains a need for a chiral/stereoisomer-specific imitation composition and a method of preparing the chiral/stereoisomer-specific imitation composition that possess improved organoleptic properties of cannabis varieties. Various embodiments described herein provide synthetic chiral compositions that include terpene and/or terpenoid enantiomers and methods for preparing synthetic chiral compositions that possesses improved organoleptic properties of cannabis varieties. Also described herein are methods for authenticating a plant cultivar comprising identifying the chiral terpene profile of the plant cultivar.

The embodiments described herein, and the various features and advantageous details thereof, are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

Unless otherwise indicated, all numbers expressing quantities of ingredients, properties such as molecular weight, reaction conditions, and so forth used in this disclosure and the appended claims are to be understood as being modified in all instances by the term “about.” Accordingly, unless indicated to the contrary, the numerical parameters set forth in this disclosure and the appended claims are approximations that may vary depending upon the desired properties sought to be obtained by the formulations. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the embodiments are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents, as used herein, are to be construed to cover both the singular and the plural, unless otherwise indicated herein or clearly contradicted by context. Recitation of ranges of values herein is merely intended to serve as a shorthand method of referring individually to each separate value failing within the range. Unless otherwise indicated herein, each individual value is incorporated into the disclosure as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate an embodiment and does not pose a limitation on the scope of the embodiment otherwise claimed. No language in the specification should be construed as indicating any non-claimed element essential to the practice of an embodiment disclosed herein.

The term “cultivar” as used herein, including the appended claims, refers to a variety of Cannabis sp. or other plants. In some embodiments, the cannabis variety is a hybrid cannabis cultivar or an inbred cannabis cultivar. The cannabis cultivar may be a variety of Cannabis sativa (Cannabis sativa L ssp sativa), Humulus lupulus, Cannabis indica (Cannabis sativa L ssp indica), or Cannabis ruderalis (Cannabis sativa L ssp ruderalis). The cannabis cultivar may be a variety selected from the group of consisting of Jack Herer #1, Purple Urkle, Diesel, Gorilla Glue #1, CBD Remedy, Girl Scout Cookies (GSC), Gorilla Glue #2, Cush #1, Blue Dream #1, Blue Dream Santa Cruz, Great White Shark San Diego, Great White Shark Humboldt, Bubblegum, Sherbert, Blueberry, Cush #2, Xj-13, Strawberry Cough, Gorilla Glue #3, Genius, Pot of Gold, Golden Pineapple, Blue Dream #2, Purple Urkle, Blue Dream #3, Jack Herer #2, and Cotton Candy, or any other accepted variety of Cannabis sp. Similarly, the Humulus sp. may be any commercially-accepted variety of hops, including Amarillo, Bravo, Cascade, Centennial, Citra, Columbus, Galena, Golding, Liberty, Mosaic, Mt. Rainier, Tahoma, Ultra, Vanguard, Williamette, and Yakima Gold. The aroma properties of the synthetic chiral composition may be detected by a human olfactory system, a human gustatory system, a synthetic nose, or any other suitable systems.

The term “modifier” as used herein, including the appended claims, refers to other classes of chemicals/compounds that are not terpenes or terpenoids. The one or more modifiers is/are added to the synthetic chiral composition along with the terpenes and the terpene enantiomers to enhance or modify the properties of the synthetic chiral composition. The one or more modifiers may be selected from the group consisting of a thiol, sulfur compound, simple hydrocarbon, ester, ketone, aldehyde, carboxylic acid, lactone, non-cannabinoid phenol, flavonoid, cannabinoid, and a combination thereof.

The terms “cannabinoid” and “cannabinoids” as used herein, including the appended claims, are interchangeable. The cannabinoid and/or cannabinoids can be selected from the group consisting of Δ9-tetrahydrocannabinol (Δ9-THC), Δ8-tetrahydrocannabinol (4Δ-THC), tetrahydrocannabinol acid (THCA), tetrahydrocannabivarin (THCV), tetrahydrocannabivarin acid (THCVA), cannabidiol (CBD), cannabidiol acid (CBDA), cannabichromene (CBC), cannabidivarin (CBDV), cannabidivarinic acid (CBDVA), cannabigerol (CBG), cannabigerol acid (CBGA), cannabigerovarin (CBGV), cannabinol (CBN), cannabinovarin (CBNV), and combination thereof.

The present disclosure provides synthetic chiral composition comprising organoleptic properties of a plant cultivar. The organoleptic properties include aroma, flavor, or other aromatic characteristics discernible by the human physiological apparatus. The specific terpene enantiomers and the additional terpenes or terpenoids impart the physiologically detectable properties to the synthetic chiral composition. The physiologically detectable properties of the plant and subsequent chiral composition can be detected and graded by a living organism capable of such analysis. The physiological detectable properties can also be analyzed via an “electronic nose.” In one embodiment, an electronic nose consists of an array of interferometers which each contain a unique olfactory receptor protein or peptide. Each interferometer protein/peptide element of the array then interacts uniquely with the detectable molecules, including differently with isomers and chiral compounds, to yield a pattern of signals that can be mapped to a biological nose and high-level scents. The same principle would also hold for an “electronic palate.”

In some embodiments described herein, the synthetic chiral composition comprises one or more isomers of at least one or more terpenes or terpenoids selected from the group consisting of alpha-bisabolol, beta-caryophyllene, borneol, camphene, camphor, delta-3-carene, caryophyllene oxide, alpha-cedrene, beta-eudesmol, fenchol, geraniol, guaiol, alpha-humulene, isoborneol, linalool, limonene, menthol, myrcene, nerol, cis-ocimene, trans-ocimene, alpha-phellandrene, alpha-pinene, beta-pinene, sabinene, alpha-terpinene, alpha-terpineol, terpinolene, alpha-guaiene, elemene, farnesene, germacrene, guaia-1(10), 11-diene, trans-2-pinanol, selina-3,7(11)-diene, eudesm-7(11)-en-4-ol, and a combination thereof.

In some embodiments described herein, the synthetic chiral composition comprises a specific ratio of one or more isomers of at least one or more terpenes or terpenoids. The total amount of one or more isomers of at least one or more terpenes or terpenoids in the synthetic chiral composition described herein may be about 0.01% by weight of the composition, about 0.02% by weight of the composition, about 0.03% by weight of the composition, about 0.04% by weight of the composition, about 0.05% by weight of the composition, about 0.1% by weight of the composition, about 0.2% by weight of the composition, about 0.3% by weight of the composition, about 0.4% by weight of the composition, about 0.5% by weight of the composition, about 1% by weight of the composition, about 1.5% by weight of the composition, about 2.0% by weight of the composition, about 2.5% by weight of the composition, about 3.0% by weight of the composition, about 3.5% by weight of the composition, about 4.0% by weight of the composition, about 4.5% by weight of the composition, about 5% by weight of the composition, about 5.5% by weight of the composition, about 6% by weight of the composition, about 6.5% by weight of the composition, about 7% by weight of the composition, about 7.5% by weight of the composition, about 8% by weight of the composition, about 8.5% by weight of the composition, about 9% by weight of the composition, about 9.5% by weight of the composition, about 10% by weight of the composition, about 10.5% by weight of the composition, about 11% by weight of the composition, about 11.5% by weight of the composition, about 12% by weight of the composition, about 12.5% by weight of the composition, about 13% by weight of the composition, about 13.5% by weight of the composition, about 14% by weight of the composition, about 14.5% by weight of the composition, about 15% by weight of the composition, about 15.5% by weight of the composition, about 16% by weight of the composition, about 16.5% by weight of the composition, about 17% by weight of the composition, about 17.5% by weight of the composition, about 18% by weight of the composition, about 18.5% by weight of the composition, about 19% by weight of the composition, about 19.5% by weight of the composition, about 20% by weight of the composition, about 20.5% by weight of the composition, about 21% by weight of the composition, about 21.5% by weight of the composition, about 22% by weight of the composition, about 22.5% by weight of the composition, about 23% by weight of the composition, about 23.5% by weight of the composition, about 24% by weight of the composition, about 24.5% by weight of the composition, about 25% by weight of the composition, about 25.5% by weight of the composition, about 26% by weight of the composition, about 26.5% by weight of the composition, about 27% by weight of the composition, about 27.5% by weight of the composition, about 28% by weight of the composition, about 28.5% by weight of the composition, about 29% by weight of the composition, about 29.5% by weight of the composition, about 30% by weight of the composition, about 30.5% by weight of the composition, about 31% by weight of the composition, about 31.5% by weight of the composition, about 32% by weight of the composition, about 32.5% by weight of the composition, about 33% by weight of the composition, about 33.5% by weight of the composition, about 34% by weight of the composition, about 34.5% by weight of the composition, about 35% by weight of the composition, about 35.5% by weight of the composition, about 36% by weight of the composition, about 36.5% by weight of the composition, about 37% by weight of the composition, about 37.5% by weight of the composition, about 38% by weight of the composition, about 38.5% by weight of the composition, about 39% by weight of the composition, about 39.5% by weight of the composition, about 40% by weight of the composition, about 40.5% by weight of the composition, about 41% by weight of the composition, about 41.5% by weight of the composition, about 42% by weight of the composition, about 42.5% by weight of the composition, about 43% by weight of the composition, about 43.5% by weight of the composition, about 44% by weight of the composition, about 44.5% by weight of the composition, about 45% by weight of the composition, about 45.5% by weight of the composition, about 46% by weight of the composition, about 46.5% by weight of the composition, about 47% by weight of the composition, about 47.5% by weight of the composition, about 48% by weight of the composition, about 48.5% by weight of the composition, about 49% by weight of the composition, about 49.5% by weight of the composition, or about 50% by weight of the composition.

In some embodiments, the synthetic chiral composition comprises one or more pinene isomers. In some embodiments, the one or more pinene isomers is selected from the group consisting of α-pinene enantiomers, β-pinene enantiomers, and a combination thereof. In one embodiment the synthetic chiral composition further comprises one or more linalool isomers. In one embodiment, the linalool isomer is an enantiomer. In yet another embodiment, the synthetic chiral composition further comprises one or more terpenes or terpenoids.

In some embodiments, the synthetic chiral composition comprises one or more pinene isomers. In some embodiments, the one or more pinene isomers can be selected from the group consisting of α-pinene enantiomers, β-pinene enantiomers, and a combination thereof. In one embodiment the synthetic chiral composition further comprises one or more linalool isomers. In one embodiment, the linalool isomer is an enantiomer. In yet another embodiment, the synthetic chiral composition further comprises at least one isomer of one or more terpenes or terpenoids.

In some embodiments described herein, the synthetic chiral composition comprises at least one pinene isomer. The total amount of one or more pinene isomers in a synthetic chiral composition described herein may range from about 0.01% to about 25% by weight of the composition. The total amount of pinene isomer(s) in a synthetic chiral composition described herein may be about 0.01% by weight of the composition, about 0.02% by weight of the composition, about 0.03% by weight of the composition, about 0.04% by weight of the composition, about 0.05% by weight of the composition, about 0.1% by weight of the composition, about 0.2% by weight of the composition, about 0.3% by weight of the composition, about 0.4% by weight of the composition, about 0.5% by weight of the composition, about 1% by weight of the composition, about 1.5% by weight of the composition, about 2.0% by weight of the composition, about 2.5% by weight of the composition, about 3.0% by weight of the composition, about 3.5% by weight of the composition, about 4.0% by weight of the composition, about 4.5% by weight of the composition, about 5% by weight of the composition, about 5.5% by weight of the composition, about 6% by weight of the composition, about 6.5% by weight of the composition, about 7% by weight of the composition, about 7.5% by weight of the composition, about 8% by weight of the composition, about 8.5% by weight of the composition, about 9% by weight of the composition, about 9.5% by weight of the composition, about 10% by weight of the composition, about 10.5% by weight of the composition, about 11% by weight of the composition, about 11.5% by weight of the composition, about 12% by weight of the composition, about 12.5% by weight of the composition, about 13% by weight of the composition, about 13.5% by weight of the composition, about 14% by weight of the composition, about 14.5% by weight of the composition, about 15% by weight of the composition, about 15.5% by weight of the composition, about 16% by weight of the composition, about 16.5% by weight of the composition, about 17% by weight of the composition, about 17.5% by weight of the composition, about 18% by weight of the composition, about 18.5% by weight of the composition, about 19% by weight of the composition, about 19.5% by weight of the composition, about 20% by weight of the composition, about 20.5% by weight of the composition, about 21% by weight of the composition, about 21.5% by weight of the composition, about 22% by weight of the composition, about 22.5% by weight of the composition, about 23% by weight of the composition, about 23.5% by weight of the composition, about 24% by weight of the composition, about 24.5% by weight of the composition, or about 25% by weight of the composition.

In one aspect of some embodiments, the at least one pinene isomer is an α-pinene isomer selected from (1R)-(+)-α-pinene and (1S)-(−)-α-pinene. The total amount of α-pinene isomer(s) in the synthetic chiral composition described herein may be about 0.01% by weight of the composition, about 0.02% by weight of the composition, about 0.03% by weight of the composition, about 0.04% by weight of the composition, about 0.05% by weight of the composition, about 0.1% by weight of the composition, about 0.2% by weight of the composition, about 0.3% by weight of the composition, about 0.4% by weight of the composition, about 0.5% by weight of the composition, about 1% by weight of the composition, about 1.5% by weight of the composition, about 2.0% by weight of the composition, about 2.5% by weight of the composition, about 3.0% by weight of the composition, about 3.5% by weight of the composition, about 4.0% by weight of the composition, about 4.5% by weight of the composition, about 5% by weight of the composition, about 5.5% by weight of the composition, about 6% by weight of the composition, about 6.5% by weight of the composition, about 7% by weight of the composition, about 7.5% by weight of the composition, about 8% by weight of the composition, about 8.5% by weight of the composition, about 9% by weight of the composition, about 9.5% by weight of the composition, about 10% by weight of the composition, about 10.5% by weight of the composition, about 11% by weight of the composition, about 11.5% by weight of the composition, about 12% by weight of the composition, about 12.5% by weight of the composition, about 13% by weight of the composition, about 13.5% by weight of the composition, about 14% by weight of the composition, about 14.5% by weight of the composition, about 15% by weight of the composition, about 15.5% by weight of the composition, about 16% by weight of the composition, about 16.5% by weight of the composition, about 17% by weight of the composition, about 17.5% by weight of the composition, about 18% by weight of the composition, about 18.5% by weight of the composition, about 19% by weight of the composition, about 19.5% by weight of the composition, about 20% by weight of the composition, about 20.5% by weight of the composition, about 21% by weight of the composition, about 21.5% by weight of the composition, about 22% by weight of the composition, about 22.5% by weight of the composition, about 23% by weight of the composition, about 23.5% by weight of the composition, about 24% by weight of the composition, about 24.5% by weight of the composition, or about 25% by weight of the composition.

In another aspect of some embodiments, the at least one pinene isomer is a β-pinene isomer selected from (1R)-(+)-β-pinene and (1S)-(−)-β-pinene. The total amount of the β-pinene isomer(s) in a synthetic chiral composition described herein may be about 0.01% by weight of the composition, about 0.02% by weight of the composition, about 0.03% by weight of the composition, about 0.04% by weight of the composition, about 0.05% by weight of the composition, about 0.1% by weight of the composition, about 0.2% by weight of the composition, about 0.3% by weight of the composition, about 0.4% by weight of the composition, about 0.5% by weight of the composition, about 1% by weight of the composition, about 1.5% by weight of the composition, about 2.0% by weight of the composition, about 2.5% by weight of the composition, about 3.0% by weight of the composition, about 3.5% by weight of the composition, about 4.0% by weight of the composition, about 4.5% by weight of the composition, about 5% by weight of the composition, about 5.5% by weight of the composition, about 6% by weight of the composition, about 6.5% by weight of the composition, about 7% by weight of the composition, about 7.5% by weight of the composition, about 8% by weight of the composition, about 8.5% by weight of the composition, about 9% by weight of the composition, about 9.5% by weight of the composition, about 10% by weight of the composition, about 10.5% by weight of the composition, about 11% by weight of the composition, about 11.5% by weight of the composition, about 12% by weight of the composition, about 12.5% by weight of the composition, about 13% by weight of the composition, about 13.5% by weight of the composition, about 14% by weight of the composition, about 14.5% by weight of the composition, about 15% by weight of the composition, about 15.5% by weight of the composition, about 16% by weight of the composition, about 16.5% by weight of the composition, about 17% by weight of the composition, about 17.5% by weight of the composition, about 18% by weight of the composition, about 18.5% by weight of the composition, about 19% by weight of the composition, about 19.5% by weight of the composition, about 20% by weight of the composition, about 20.5% by weight of the composition, about 21% by weight of the composition, about 21.5% by weight of the composition, about 22% by weight of the composition, about 22.5% by weight of the composition, about 23% by weight of the composition, about 23.5% by weight of the composition, about 24% by weight of the composition, about 24.5% by weight of the composition, or about 25% by weight of the composition.

In yet another aspect of some embodiments, the at least one pinene isomer includes at least one α-pinene isomer and at least one β-pinene isomer. The total amount of the at least one α-pinene isomer and the at least one β-pinene isomer in a synthetic chiral composition described herein may be about 0.01% by weight of the composition, about 0.02% by weight of the composition, about 0.03% by weight of the composition, about 0.04% by weight of the composition, about 0.05% by weight of the composition, about 0.1% by weight of the composition, about 0.2% by weight of the composition, about 0.3% by weight of the composition, about 0.4% by weight of the composition, about 0.5% by weight of the composition, about 1% by weight of the composition, about 1.5% by weight of the composition, about 2.0% by weight of the composition, about 2.5% by weight of the composition, about 3.0% by weight of the composition, about 3.5% by weight of the composition, about 4.0% by weight of the composition, about 4.5% by weight of the composition, about 5% by weight of the composition, about 5.5% by weight of the composition, about 6% by weight of the composition, about 6.5% by weight of the composition, about 7% by weight of the composition, about 7.5% by weight of the composition, about 8% by weight of the composition, about 8.5% by weight of the composition, about 9% by weight of the composition, about 9.5% by weight of the composition, about 10% by weight of the composition, about 10.5% by weight of the composition, about 11% by weight of the composition, about 11.5% by weight of the composition, about 12% by weight of the composition, about 12.5% by weight of the composition, about 13% by weight of the composition, about 13.5% by weight of the composition, about 14% by weight of the composition, about 14.5% by weight of the composition, about 15% by weight of the composition, about 15.5% by weight of the composition, about 16% by weight of the composition, about 16.5% by weight of the composition, about 17% by weight of the composition, about 17.5% by weight of the composition, about 18% by weight of the composition, about 18.5% by weight of the composition, about 19% by weight of the composition, about 19.5% by weight of the composition, about 20% by weight of the composition, about 20.5% by weight of the composition, about 21% by weight of the composition, about 21.5% by weight of the composition, about 22% by weight of the composition, about 22.5% by weight of the composition, about 23% by weight of the composition, about 23.5% by weight of the composition, about 24% by weight of the composition, about 24.5% by weight of the composition, about 25% by weight of the composition, about 25.5% by weight of the composition, about 0.526% by weight of the composition, about 26.5% by weight of the composition, about 27% by weight of the composition, about 27.5% by weight of the composition, about 28% by weight of the composition, about 28.5% by weight of the composition, about 29% by weight of the composition, about 29.5% by weight of the composition, about 30% by weight of the composition, about 30.5% by weight of the composition, about 31% by weight of the composition, about 31.5% by weight of the composition, about 32% by weight of the composition, about 32.5% by weight of the composition, about 33% by weight of the composition, about 33.5% by weight of the composition, about 34% by weight of the composition, about 34.5% by weight of the composition, about 35% by weight of the composition, about 35.5% by weight of the composition, about 36% by weight of the composition, about 36.5% by weight of the composition, about 37% by weight of the composition, about 37.5% by weight of the composition, about 38% by weight of the composition, about 38.5% by weight of the composition, about 39% by weight of the composition, about 39.5% by weight of the composition, about 40% by weight of the composition, about 40.5% by weight of the composition, about 41% by weight of the composition, about 41.5% by weight of the composition, about 42% by weight of the composition, about 42.5% by weight of the composition, about 43% by weight of the composition, about 43.5% by weight of the composition, about 44% by weight of the composition, about 44.5% by weight of the composition, about 45% by weight of the composition, about 45.5% by weight of the composition, about 46% by weight of the composition, about 46.5% by weight of the composition, about 47% by weight of the composition, about 47.5% by weight of the composition, about 48% by weight of the composition, about 48.5% by weight of the composition, about 49% by weight of the composition, about 49.5% by weight of the composition, or about 50% by weight of the composition.

In some embodiments described herein, the synthetic chiral composition comprises at least one linalool isomer. In some embodiments, the linalool isomer is a linalool enantiomer. In one aspect of some embodiments, the at least one linalool isomer is selected from (S)-(+)-linalool and (R)-(−)-linalool. The total amount of the at least one linalool isomer in a synthetic chiral composition described herein may range from about 0.01% to about 25% by weight of the composition. The total amount of linalool isomers in the synthetic chiral composition described herein may be about 0.01% by weight of the composition, about 0.02% by weight of the composition, about 0.03% by weight of the composition, about 0.04% by weight of the composition, about 0.05% by weight of the composition, about 0.1% by weight of the composition, about 0.2% by weight of the composition, about 0.3% by weight of the composition, about 0.4% by weight of the composition, about 0.5% by weight of the composition, about 1% by weight of the composition, about 1.5% by weight of the composition, about 2.0% by weight of the composition, about 2.5% by weight of the composition, about 3.0% by weight of the composition, about 3.5% by weight of the composition, about 4.0% by weight of the composition, about 4.5% by weight of the composition, about 5% by weight of the composition, about 5.5% by weight of the composition, about 6% by weight of the composition, about 6.5% by weight of the composition, about 7% by weight of the composition, about 7.5% by weight of the composition, about 8% by weight of the composition, about 8.5% by weight of the composition, about 9% by weight of the composition, about 9.5% by weight of the composition, about 10% by weight of the composition, about 10.5% by weight of the composition, about 11% by weight of the composition, about 11.5% by weight of the composition, about 12% by weight of the composition, about 12.5% by weight of the composition, about 13% by weight of the composition, about 13.5% by weight of the composition, about 14% by weight of the composition, about 14.5% by weight of the composition, about 15% by weight of the composition, about 15.5% by weight of the composition, about 16% by weight of the composition, about 16.5% by weight of the composition, about 17% by weight of the composition, about 17.5% by weight of the composition, about 18% by weight of the composition, about 18.5% by weight of the composition, about 19% by weight of the composition, about 19.5% by weight of the composition, about 20% by weight of the composition, about 20.5% by weight of the composition, about 21% by weight of the composition, about 21.5% by weight of the composition, about 22% by weight of the composition, about 22.5% by weight of the composition, about 23% by weight of the composition, about 23.5% by weight of the composition, about 24% by weight of the composition, about 24.5% by weight of the composition, or about 25% by weight of the composition.

In some embodiments described herein, the synthetic chiral composition comprises one or more terpenes or terpenoids selected from the group consisting of alpha-bisabolol, beta-caryophyllene, borneol, camphene, camphor, delta-3-carene, caryophyllene oxide, alpha-cedrene, beta-eudesmol, fenchol, geraniol, guaiol, alpha-humulene, isoborneol, limonene, menthol, myrcene, nerol, cis-ocimene, trans-ocimene, alpha-phellandrene, sabinene, alpha-terpinene, alpha-terpineol, terpinolene, alpha-guaiene, elemene, farnesene, germacrene, guaia-1(10), 11-diene, trans-2-pinanol, selina-3,7(11)-diene, eudesm-7(11)-en-4-ol, and a combination thereof. The total amount of one or more terpenes or terpenoids in the synthetic chiral composition described herein may range from 0.01% to 99% by weight of the composition. The total amount of one or more terpenes or terpenoids in the synthetic chiral composition described herein may be about 0.01% by weight of the composition, about 0.02% by weight of the composition, about 0.03% by weight of the composition, about 0.04% by weight of the composition, about 0.05% by weight of the composition, about 0.1% by weight of the composition, about 0.2% by weight of the composition, about 0.3% by weight of the composition, about 0.4% by weight of the composition, about 0.5% by weight of the composition, about 1.0% by weight of the composition, about 2% by weight of the composition, about 3% by weight of the composition, about 4% by weight of the composition, about 5% by weight of the composition, about 6% by weight of the composition, about 7% by weight of the composition, about 8% by weight of the composition, about 9% by weight of the composition, about 10% by weight of the composition, about 11% by weight of the composition, about 12% by weight of the composition, about 13% by weight of the composition, about 14% by weight of the composition, about 15% by weight of the composition, about 16% by weight of the composition, about 17% by weight of the composition, about 18% by weight of the composition, about 19% by weight of the composition, about 20% by weight of the composition, about 21% by weight of the composition, about 22% by weight of the composition, about 23% by weight of the composition, about 24% by weight of the composition, about 25% by weight of the composition, about 26% by weight of the composition, about 27% by weight of the composition, about 28% by weight of the composition, about 29% by weight of the composition, about 30% by weight of the composition, about 31% by weight of the composition, about 32% by weight of the composition, about 33% by weight of the composition, about 34% by weight of the composition, about 35% by weight of the composition, about 36% by weight of the composition, about 37% by weight of the composition, about 38% by weight of the composition, about 39% by weight of the composition, about 40% by weight of the composition, about 41% by weight of the composition, about 42% by weight of the composition, about 43% by weight of the composition, about 44% by weight of the composition, about 45% by weight of the composition, about 46% by weight of the composition, about 47% by weight of the composition, about 48% by weight of the composition, about 49% by weight of the composition, about 50% by weight of the composition, about 51% by weight of the composition, about 52% by weight of the composition, about 53% by weight of the composition, about 54% by weight of the composition, about 55% by weight of the composition, about 56% by weight of the composition, about 57% by weight of the composition, about 58% by weight of the composition, about 59% by weight of the composition, about 60% by weight of the composition, about 61% by weight of the composition, about 62% by weight of the composition, about 63% by weight of the composition, about 64% by weight of the composition, about 65% by weight of the composition, about 66% by weight of the composition, about 67% by weight of the composition, about 68% by weight of the composition, about 69% by weight of the composition, about 70% by weight of the composition, about 71% by weight of the composition, about 72% by weight of the composition, about 73% by weight of the composition, about 74% by weight of the composition, about 75% by weight of the composition, about 76% by weight of the composition, about 77% by weight of the composition, about 78% by weight of the composition, about 79% by weight of the composition, about 80% by weight of the composition, about 81% by weight of the composition, about 82% by weight of the composition, about 83% by weight of the composition, about 84% by weight of the composition, about 85% by weight of the composition, about 86% by weight of the composition, about 87% by weight of the composition, about 88% by weight of the composition, about 89% by weight of the composition, about 90% by weight of the composition, about 91% by weight of the composition, about 92% by weight of the composition, about 93% by weight of the composition, about 94% by weight of the composition, about 95% by weight of the composition, about 96% by weight of the composition, about 97% by weight of the composition, about 98% by weight of the composition, or about 99% by weight of the composition.

In some embodiments described herein, the one or more terpenes or terpenoids in the synthetic chiral composition may be one or more isomers of terpenes or terpenoids selected from the group consisting of alpha-bisabolol, borneol, camphene, camphor, delta-3-carene, caryophyllene oxide, alpha-cedrene, beta-eudesmol, fenchol, geraniol, guaiol, alpha-humulene, isoborneol, menthol, myrcene, nerol, cis-ocimene, trans-ocimene, alpha- phellandrene, sabinene, alpha-terpinene, alpha-terpineol, terpinolene, alpha-guaiene, elemene, farnesene, germacrene, guaia-1(10), 11-diene, trans-2-pinanol, selina-3,7(11)-diene, eudesm-7(11)-en-4-ol, valencene, and a combination thereof. The total amount of one or more isomers, when isomers exist, of the terpenes or terpenoids in the synthetic chiral composition described herein may range from 0.01% to 99% by weight of the composition. The total amount of one or more isomers of the terpenes or terpenoids in the synthetic chiral composition described herein may be about 0.01% by weight of the composition, about 0.02% by weight of the composition, about 0.03% by weight of the composition, about 0.04% by weight of the composition, about 0.05% by weight of the composition, about 0.1% by weight of the composition, about 0.2% by weight of the composition, about 0.3% by weight of the composition, about 0.4% by weight of the composition, about 0.5% by weight of the composition, about 1.0% by weight of the composition, about 2% by weight of the composition, about 3% by weight of the composition, about 4% by weight of the composition, about 5% by weight of the composition, about 6% by weight of the composition, about 7% by weight of the composition, about 8% by weight of the composition, about 9% by weight of the composition, about 10% by weight of the composition, about 11% by weight of the composition, about 12% by weight of the composition, about 13% by weight of the composition, about 14% by weight of the composition, about 15% by weight of the composition, about 16% by weight of the composition, about 17% by weight of the composition, about 18% by weight of the composition, about 19% by weight of the composition, about 20% by weight of the composition, about 21% by weight of the composition, about 22% by weight of the composition, about 23% by weight of the composition, about 24% by weight of the composition, about 25% by weight of the composition, about 26% by weight of the composition, about 27% by weight of the composition, about 28% by weight of the composition, about 29% by weight of the composition, about 30% by weight of the composition, about 31% by weight of the composition, about 32% by weight of the composition, about 33% by weight of the composition, about 34% by weight of the composition, about 35% by weight of the composition, about 36% by weight of the composition, about 37% by weight of the composition, about 38% by weight of the composition, about 39% by weight of the composition, about 40% by weight of the composition, about 41% by weight of the composition, about 42% by weight of the composition, about 43% by weight of the composition, about 44% by weight of the composition, about 45% by weight of the composition, about 46% by weight of the composition, about 47% by weight of the composition, about 48% by weight of the composition, about 49% by weight of the composition, about 50% by weight of the composition, about 51% by weight of the composition, about 52% by weight of the composition, about 53% by weight of the composition, about 54% by weight of the composition, about 55% by weight of the composition, about 56% by weight of the composition, about 57% by weight of the composition, about 58% by weight of the composition, about 59% by weight of the composition, about 60% by weight of the composition, about 61% by weight of the composition, about 62% by weight of the composition, about 63% by weight of the composition, about 64% by weight of the composition, about 65% by weight of the composition, about 66% by weight of the composition, about 67% by weight of the composition, about 68% by weight of the composition, about 69% by weight of the composition, about 70% by weight of the composition, about 71% by weight of the composition, about 72% by weight of the composition, about 73% by weight of the composition, about 74% by weight of the composition, about 75% by weight of the composition, about 76% by weight of the composition, about 77% by weight of the composition, about 78% by weight of the composition, about 79% by weight of the composition, about 80% by weight of the composition, about 81% by weight of the composition, about 82% by weight of the composition, about 83% by weight of the composition, about 84% by weight of the composition, about 85% by weight of the composition, about 86% by weight of the composition, about 87% by weight of the composition, about 88% by weight of the composition, about 89% by weight of the composition, about 90% by weight of the composition, about 91% by weight of the composition, about 92% by weight of the composition, about 93% by weight of the composition, about 94% by weight of the composition, about 95% by weight of the composition, about 96% by weight of the composition, about 97% by weight of the composition, about 98% by weight of the composition, or about 99% by weight of the composition.

In some embodiments described herein, the synthetic chiral composition comprises one or more modifiers. The total amount of one or more modifiers in the synthetic chiral composition described herein may range from 0.01% to 90% by weight of the composition. The total amount of one or more modifiers in the synthetic chiral composition described herein may be about 0.01% by weight of the composition, about 0.02% by weight of the composition, about 0.03% by weight of the composition, about 0.04% by weight of the composition, about 0.05% by weight of the composition, about 0.1% by weight of the composition, about 0.2% by weight of the composition, about 0.3% by weight of the composition, about 0.4% by weight of the composition, about 0.5% by weight of the composition, about 1.0% by weight of the composition, about 2% by weight of the composition, about 3% by weight of the composition, about 4% by weight of the composition, about 5% by weight of the composition, about 6% by weight of the composition, about 7% by weight of the composition, about 8% by weight of the composition, about 9% by weight of the composition, about 10% by weight of the composition, about 11% by weight of the composition, about 12% by weight of the composition, about 13% by weight of the composition, about 14% by weight of the composition, about 15% by weight of the composition, about 16% by weight of the composition, about 17% by weight of the composition, about 18% by weight of the composition, about 19% by weight of the composition, about 20% by weight of the composition, about 21% by weight of the composition, about 22% by weight of the composition, about 23% by weight of the composition, about 24% by weight of the composition, about 25% by weight of the composition, about 26% by weight of the composition, about 27% by weight of the composition, about 28% by weight of the composition, about 29% by weight of the composition, about 30% by weight of the composition, about 31% by weight of the composition, about 32% by weight of the composition, about 33% by weight of the composition, about 34% by weight of the composition, about 35% by weight of the composition, about 36% by weight of the composition, about 37% by weight of the composition, about 38% by weight of the composition, about 39% by weight of the composition, about 40% by weight of the composition, about 41% by weight of the composition, about 42% by weight of the composition, about 43% by weight of the composition, about 44% by weight of the composition, about 45% by weight of the composition, about 46% by weight of the composition, about 47% by weight of the composition, about 48% by weight of the composition, about 49% by weight of the composition, about 50% by weight of the composition, about 51% by weight of the composition, about 52% by weight of the composition, about 53% by weight of the composition, about 54% by weight of the composition, about 55% by weight of the composition, about 56% by weight of the composition, about 57% by weight of the composition, about 58% by weight of the composition, about 59% by weight of the composition, about 60% by weight of the composition, about 61% by weight of the composition, about 62% by weight of the composition, about 63% by weight of the composition, about 64% by weight of the composition, about 65% by weight of the composition, about 66% by weight of the composition, about 67% by weight of the composition, about 68% by weight of the composition, about 69% by weight of the composition, about 70% by weight of the composition, about 71% by weight of the composition, about 72% by weight of the composition, about 73% by weight of the composition, about 74% by weight of the composition, about 75% by weight of the composition, about 76% by weight of the composition, about 77% by weight of the composition, about 78% by weight of the composition, about 79% by weight of the composition, about 80% by weight of the composition, about 81% by weight of the composition, about 82% by weight of the composition, about 83% by weight of the composition, about 84% by weight of the composition, about 85% by weight of the composition, about 86% by weight of the composition, about 87% by weight of the composition, about 88% by weight of the composition, about 89% by weight of the composition, or about 90% by weight of the composition.

In some embodiments, the pinene isomers in a synthetic chiral composition described herein are purified from a natural source. In another embodiment described herein, the pinene isomers in a synthetic chiral composition are produced synthetically.

In some embodiments, the linalool isomers in a synthetic chiral composition described herein are purified from a natural source. In another embodiment described herein, the linalool isomers in a synthetic chiral composition are produced synthetically.

In some embodiments, the terpenes or terpenoids, and/or isomers thereof, in a synthetic chiral composition described herein are purified from a natural source. In another embodiment described herein, the terpenes or terpenoids, and/or isomers thereof, in a synthetic chiral composition are produced synthetically.

In some embodiments described herein, the synthetic chiral composition includes a natural form, a synthetic form, or a combination of natural and synthetic form of the pinene isomers, the linalool isomers, and/or the terpenes or terpenoids.

The present disclosure also provides formulations comprising: (i) a synthetic chiral composition described herein and (ii) at least one or more additional terpenes or terpenoids selected from the group consisting of alpha-bisabolol, borneol, camphene, camphor, delta-3-carene, caryophyllene oxide, alpha-cedrene, beta-eudesmol, fenchol, geraniol, guaiol, alpha-humulene, isoborneol, linalool, menthol, nerol, cis-ocimene, trans-ocimene, alpha-phellandrene, alpha-pinene, beta-pinene, sabinene, alpha-terpinene, alpha-terpineol, terpinolene, alpha-gauaiene, elemene, farnesene, germacrene, guaia-1(10),11-diene, trans-2-pinanol, Selina-3,7(11)-diene, eudesm-7(11)-en-4-ol, valencene, and a combination thereof.

In some embodiments, the formulation comprises at least 2% (wt./vol.) to at least 99% (wt./vol.) of a synthetic chiral composition described herein. In some embodiments, the formulation comprises about 2% (wt./vol.), about 3% (wt./vol.), about 4% (wt./vol.), about 5% (wt./vol.), about 6% (wt./vol.), about 7% (wt./vol.), about 8% (wt./vol.), about 9% (wt./vol.), about 10% (wt./vol.), about 11% (wt./vol.), about 12% (wt./vol.), about 13% (wt./vol.), about 14% (wt./vol.), about 15% (wt./vol.), about 16% (wt./vol.), about 17% (wt./vol.), about 18% (wt./vol.), about 19% (wt./vol.), about 20% (wt./vol.), about 21% (wt./vol.), about 22% (wt./vol.), about 23% (wt./vol.), about 24% (wt./vol.), about 25% (wt./vol.), about 26% (wt./vol.), about 27% (wt./vol.), about 28% (wt./vol.), about 28% (wt./vol.), about 29% (wt./vol.), about 30% (wt./vol.), about 31% (wt./vol.), about 32% (wt./vol.), about 33% (wt./vol.), about 34% (wt./vol.), about 35% (wt./vol.), about 36% (wt./vol.), about 37% (wt./vol.), about 38% (wt./vol.), about 39% (wt./vol.), about 40% (wt./vol.), about 41% (wt./vol.), about 42% (wt./vol.), about 43% (wt./vol.), about 44% (wt./vol.), about 45% (wt./vol.), about 46% (wt./vol.), about 47% (wt./vol.), about 48% (wt./vol.), about 49% (wt./vol.), about 50% (wt./vol.), about 51% (wt./vol.), about 52% (wt./vol.), about 53% (wt./vol.), about 54% (wt./vol.), about 55% (wt./vol.), about 56% (wt./vol.), about 57% (wt./vol.), about 58% (wt./vol.), about 59% (wt./vol.), about 60% (wt./vol.), about 61% (wt./vol.), about 62% (wt./vol.), about 63% (wt./vol.), about 64% (wt./vol.), about 65% (wt./vol.), about 66% (wt./vol.), about 67% (wt./vol.), about 68% (wt./vol.), about 69% (wt./vol.), about 70% (wt./vol.), about 71% (wt./vol.), about 72% (wt./vol.), about 73% (wt./vol.), about 74% (wt./vol.), about 75% (wt./vol.), about 76% (wt./vol.), about 77% (wt./vol.), about 78% (wt./vol.), about 79% (wt./vol.), about 80% (wt./vol.), about 81% (wt./vol.), about 82% (wt./vol.), about 83% (wt./vol.), about 84% (wt./vol.), about 85% (wt./vol.), about 86% (wt./vol.), about 87% (wt./vol.), about 88% (wt./vol.), about 89% (wt./vol.), about 90% (wt./vol.), about 91% (wt./vol.), about 92% (wt./vol.), about 93% (wt./vol.), about 94% (wt./vol.), about 95% (wt./vol.), about 96% (wt./vol.), about 97% (wt./vol.), about 98% (wt./vol.), or about 99% (wt./vol.) of a synthetic chiral composition described herein.

In some embodiments, the formulation comprises at least 2% (wt./vol.) to at least 99% (wt./vol.) of the at least one or more additional terpenes or terpenoids. In some embodiments, the formulation comprises about 2% (wt./vol.), about 3% (wt./vol.), about 4% (wt./vol.), about 5% (wt./vol.), about 6% (wt./vol.), about 7% (wt./vol.), about 8% (wt./vol.), about 9% (wt./vol.), about 10% (wt./vol.), about 11% (wt./vol.), about 12% (wt./vol.), about 13% (wt./vol.), about 14% (wt./vol.), about 15% (wt./vol.), about 16% (wt./vol.), about 17% (wt./vol.), about 18% (wt./vol.), about 19% (wt./vol.), about 20% (wt./vol.), about 21% (wt./vol.), about 22% (wt./vol.), about 23% (wt./vol.), about 24% (wt./vol.), about 25% (wt./vol.), about 26% (wt./vol.), about 27% (wt./vol.), about 28% (wt./vol.), about 28% (wt./vol.), about 29% (wt./vol.), about 30% (wt./vol.), about 31% (wt./vol.), about 32% (wt./vol.), about 33% (wt./vol.), about 34% (wt./vol.), about 35% (wt./vol.), about 36% (wt./vol.), about 37% (wt./vol.), about 38% (wt./vol.), about 39% (wt./vol.), about 40% (wt./vol.), about 41% (wt./vol.), about 42% (wt./vol.), about 43% (wt./vol.), about 44% (wt./vol.), about 45% (wt./vol.), about 46% (wt./vol.), about 47% (wt./vol.), about 48% (wt./vol.), about 49% (wt./vol.), about 50% (wt./vol.), about 51% (wt./vol.), about 52% (wt./vol.), about 53% (wt./vol.), about 54% (wt./vol.), about 55% (wt./vol.), about 56% (wt./vol.), about 57% (wt./vol.), about 58% (wt./vol.), about 59% (wt./vol.), about 60% (wt./vol.), about 61% (wt./vol.), about 62% (wt./vol.), about 63% (wt./vol.), about 64% (wt./vol.), about 65% (wt./vol.), about 66% (wt./vol.), about 67% (wt./vol.), about 68% (wt./vol.), about 69% (wt./vol.), about 70% (wt./vol.), about 71% (wt./vol.), about 72% (wt./vol.), about 73% (wt./vol.), about 74% (wt./vol.), about 75% (wt./vol.), about 76% (wt./vol.), about 77% (wt./vol.), about 78% (wt./vol.), about 79% (wt./vol.), about 80% (wt./vol.), about 81% (wt./vol.), about 82% (wt./vol.), about 83% (wt./vol.), about 84% (wt./vol.), about 85% (wt./vol.), about 86% (wt./vol.), about 87% (wt./vol.), about 88% (wt./vol.), about 89% (wt./vol.), about 90% (wt./vol.), about 91% (wt./vol.), about 92% (wt./vol.), about 93% (wt./vol.), about 94% (wt./vol.), about 95% (wt./vol.), about 96% (wt./vol.), about 97% (wt./vol.), about 98% (wt./vol.), or about 99% (wt./vol.) of the at least one or more additional terpenes or terpenoids.

As used herein, the phrase “knowledge base” means a structured dataset of cultivar samples, cultivar names, the chemical constituents found in the sample, and the amount of the chemical constituents found. In some knowledge base embodiments, the chemical constituents and the amount thereof can be stored in the form of peak table, which connects the chromatogram to the constituent peaks and the amounts thereof. In some other database embodiments, the characterizing data stored is in the form of whole chromatograms which imply the chemical constituents and amounts thereof and can be used for pattern-matching operations.

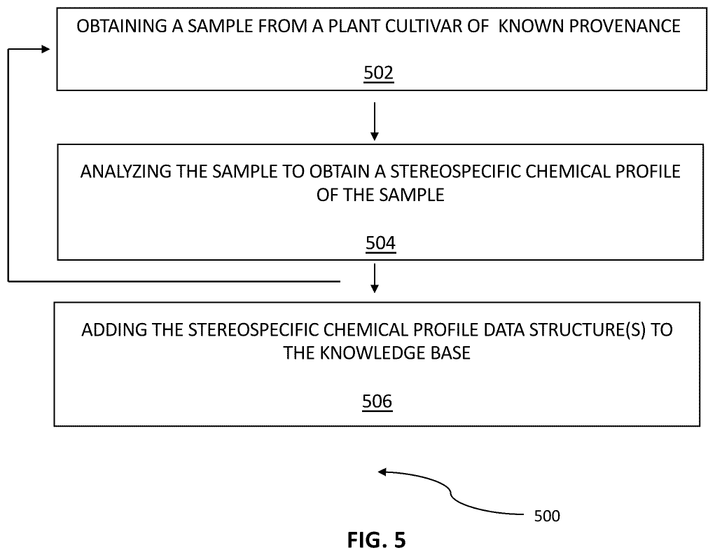

FIG. 5 depicts a recursive flow diagram 500 of the process of creating a knowledge base. At step 502 a sample is obtained from a plant cultivar of known provenance. At step 504 the sample is analyzed to obtain a stereospecific chemical profile of the sample. Steps 502 and 504 are repeated for all plant samples to be analyzed. At step 506 the stereospecific chemical profile data structures determined by step 504 are added to the knowledge base.

The knowledge base as described above can be queried with analytical data produced via the analysis of an unknown sample. In this example, the unknown sample is an unknown cannabis sample, called Sample U, and the database is a knowledge base containing a multitude of previous entries for positively-identified cannabis samples. The analytical data, or alternatively the entire chromatogram, for Sample U is then compared to each database entry. The comparison produces a similarity score. Above a certain similarity threshold, Sample U can be positively identified to be the same cannabis cultivar as the corresponding entry in the database. If the similarity threshold is not reached for any entry, then Sample U is a new cultivar not currently represented in the database.

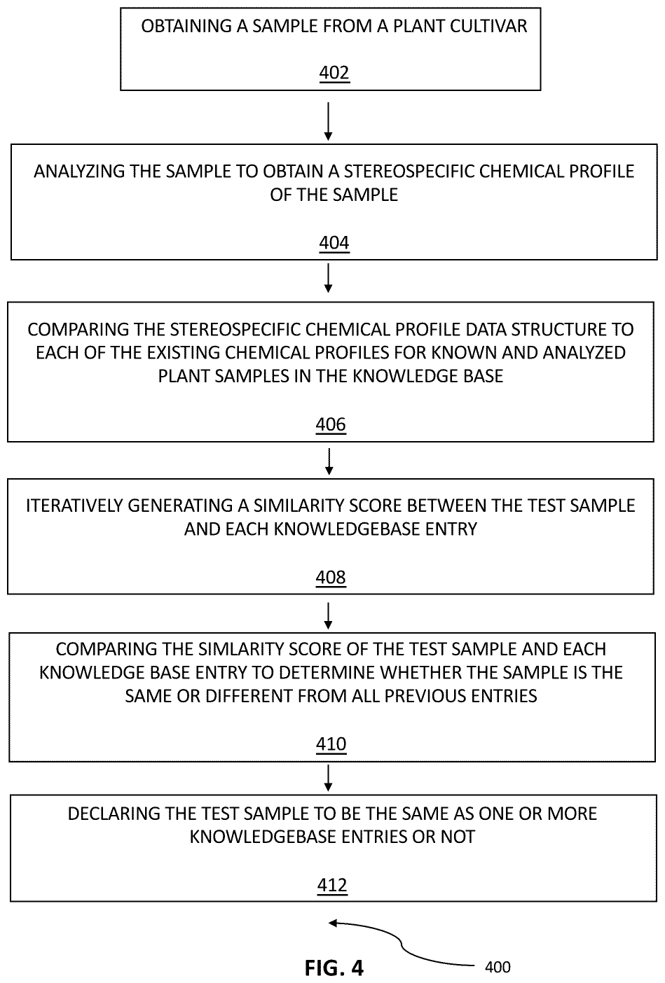

FIG. 4 depicts a flow diagram 400 of this process. At step 402 a sample is obtained from a plant cultivar. At step 404 the sample is analyzed to obtain a stereospecific chemical profile. At step 406 the profile data structure is compared to each of the existing chemical profiles for known and analyzed plant samples in the knowledge base. At step 408 a similarity score is iteratively generated between the test sample and each knowledge base entry. At step 410 the similarity score of the test sample and each knowledge base entry is compared to determine is the same or different from all previous entries. At step 412, after the comparison, the test sample is declared to either be the same as one or more knowledge based entries or not.

An embodiment further discloses a formulation comprising: (i) at least one alpha-pinene enantiomer, (ii) at least one beta-pinene enantiomer, (iii) at least one linalool enantiomer, and (iv) one or more additional terpenes or terpenoids selected from the group consisting of myrcene, beta-caryophyllene, limonene, alpha-bisabolol, borneol, camphene, camphor, delta-3-carene, caryophyllene oxide, alpha-cedrene, beta-eudesmol, fenchol, geraniol, guaiol, alpha-humulene, isoborneol, linalool, menthol, nerol, cis-ocimene, trans-ocimene, alpha-phellandrene, alpha-pinene, beta-pinene, sabinene, alpha-terpinene, alpha-terpineol, terpinolene, alpha-gauaiene, elemene, farnesene, germacrene, guaia-1(10),11-diene, trans-2-pinanol, selina-3,7(11)-diene, eudesm-7(11)-en-4-ol, valencene and a combination thereof.

In some embodiments, the at least one alpha-pinene enantiomer, the at least one beta-pinene enantiomer, the at least one linalool enantiomer and the one or more terpenes or terpenoids together include in a range of about 2% weight/volume (wt./vol.) to 98% (wt./vol.) of the synthetic chiral formulation. In some embodiments, the amount of the at least one alpha-pinene enantiomer, the at least one beta-pinene enantiomer, the at least one linalool enantiomer and the one or more terpenes or terpenoids together is about 2% (wt./vol.), about 3% (wt./vol.), about 4% (wt./vol.), about 5% (wt./vol.), about 6% (wt./vol.), about 7% (wt./vol.), about 8% (wt./vol.), about 9% (wt./vol.), about 10% (wt./vol.), about 11% (wt./vol.), about 12% (wt./vol.), about 13% (wt./vol.), about 14% (wt./vol.), about 15% (wt./vol.), about 16% (wt./vol.), about 17% (wt./vol.), about 18% (wt./vol.), about 19% (wt./vol.), about 20% (wt./vol.), about 21% (wt./vol.), about 22% (wt./vol.), about 23% (wt./vol.), about 24% (wt./vol.), about 25% (wt./vol.), about 26% (wt./vol.), about 27% (wt./vol.), about 28% (wt./vol.), about 28% (wt./vol.), about 29% (wt./vol.), about 30% (wt./vol.), about 31% (wt./vol.), about 32% (wt./vol.), about 33% (wt./vol.), about 34% (wt./vol.), about 35% (wt./vol.), about 36% (wt./vol.), about 37% (wt./vol.), about 38% (wt./vol.), about 39% (wt./vol.), about 40% (wt./vol.), about 41% (wt./vol.), about 42% (wt./vol.), about 43% (wt./vol.), about 44% (wt./vol.), about 45% (wt./vol.), about 46% (wt./vol.), about 47% (wt./vol.), about 48% (wt./vol.), about 49% (wt./vol.), about 50% (wt./vol.), about 51% (wt./vol.), about 52% (wt./vol.), about 53% (wt./vol.), about 54% (wt./vol.), about 55% (wt./vol.), about 56% (wt./vol.), about 57% (wt./vol.), about 58% (wt./vol.), about 59% (wt./vol.), about 60% (wt./vol.), about 61% (wt./vol.), about 62% (wt./vol.), about 63% (wt./vol.), about 64% (wt./vol.), about 65% (wt./vol.), about 66% (wt./vol.), about 67% (wt./vol.), about 68% (wt./vol.), about 69% (wt./vol.), about 70% (wt./vol.), about 71% (wt./vol.), about 72% (wt./vol.), about 73% (wt./vol.), about 74% (wt./vol.), about 75% (wt./vol.), about 76% (wt./vol.), about 77% (wt./vol.), about 78% (wt./vol.), about 79% (wt./vol.), about 80% (wt./vol.), about 81% (wt./vol.), about 82% (wt./vol.), about 83% (wt./vol.), about 84% (wt./vol.), about 85% (wt./vol.), about 86% (wt./vol.), about 87% (wt./vol.), about 88% (wt./vol.), about 89% (wt./vol.), about 90% (wt./vol.), about 91% (wt./vol.), about 92% (wt./vol.), about 93% (wt./vol.), about 94% (wt./vol.), about 95% (wt./vol.), about 96% (wt./vol.), about 97% (wt./vol.), or about 98% (wt./vol.) of the synthetic chiral formulation.

In some embodiments, a ratio of (1R)-(+)-α-pinene to (1S)-(−)-α-pinene in the synthetic chiral formulation is ranging from 100:1 to 1:100. In some embodiments, a ratio of (1R)-(+)-β-pinene to (1S)-(−)-β-pinene in the synthetic chiral formulation is ranging from 100:1to 1:100. In some embodiments, a ratio of (S)-(+)-linalool to (R)-(−)-linalool in the synthetic chiral formulation is ranging from 100:1 to 1:100.

In some embodiments, the one or more terpenes or terpenoids in the formulation is myrcene. In some embodiments, the one or more terpenes or terpenoids in the formulation is beta-caryophyllene. In some embodiments, the one or more terpenes or terpenoids in the formulation is limonene. In some embodiments, the one or more terpenes or terpenoids is terpinolene. In some embodiments, the one or more terpenes or terpenoids in the formulation is beta-caryophyllene and limonene. In some embodiments, the one or more terpenes or terpenoids in the formulation is beta-caryophyllene and terpinolene. In some embodiments, the one or more terpenes or terpenoids in the formulation is not myrcene. In some embodiments, the one or more terpenes or terpenoids in the formulation is not (S)-(−)-limonene.

The formulation further includes one or more modifiers that are selected from a group consisting of a thiol, sulfur compound, simple hydrocarbon, ester, ketone, aldehyde, carboxylic acid, lactone, non-cannabinoid phenol, flavonoid, cannabinoid, and a combination thereof. In some embodiments, the one or more modifiers in the synthetic chiral formulation included are present in a range of about 0.5% (wt./vol.) to 90% (wt./vol.). In some embodiments, the formulation does not include any essential oils. In some embodiments, the formulation does not include water.

In some embodiments, the formulation further includes one or more inert compounds. The one or more inert compounds may include, but are not limited to, excipients, viscosity-imparting agents, solvents, binders, lubricants, stabilizers, preservatives, diluents, and the like. In some embodiments, the formulation may be made into a liquid, a slurry, an emulsion, a suspension, a gel, and the like.

In some embodiments, the formulation may be a perfume, an incense, a cosmetic, a moisturizer, an emollient, a toiletry, an edible substance, an inhalable substance, an e-cigarette liquid, a candle and the like. In some embodiments, the formulation may be included in an apparatus. The apparatus may include, but not limited to, a container that contains perfume or soap or cosmetics or spray composition for aerosol dispersion and other fragrance materials, a scratch and sniff device, an electronic cigarette, a wax candle, an edible substance, an insect repellent or attractant device and the like.

The formulations disclosed herein can be prepared by combining terpene enantiomers (i.e. alpha-pinene enantiomers, beta-pinene enantiomers, and linalool enantiomers) with one or more terpenes or terpenoids that are selected from a group consisting of myrcene, beta-caryophyllene, limonene, alpha-bisabolol, borneol, camphene, camphor, delta-3-carene, caryophyllene oxide, alpha-cedrene, beta-eudesmol, fenchol, geraniol, guaiol, alpha-humulene, isoborneol, linalool, menthol, nerol, cis-ocimene, trans-ocimene, alpha-phellandrene, alpha-pinene, beta-pinene, sabinene, alpha-terpinene, alpha-terpineol, terpinolene, alpha-gauaiene, elemene, farnesene, germacrene, guaia-1(10),11-diene, trans-2-pinanol, selina-3,7(11)-diene, eudesm-7(11)-en-4-ol, valencene, and a combination thereof.

### EXAMPLES

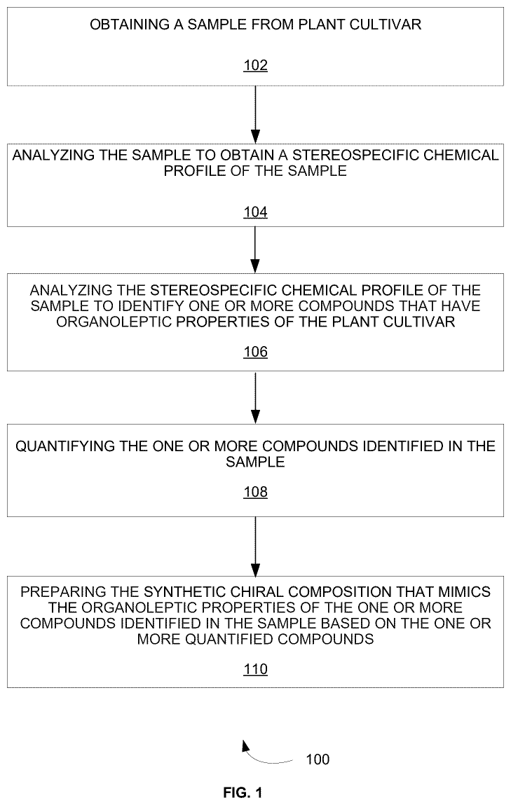

Referring now to the drawings, and more particularly to FIGS. 1 through 3, and the examples highlighted below, specific embodiments of the present disclosure were carried out:

EXAMPLE-1—Preparation of synthetic chiral composition: The knowledge base described above containing chiral analytical data for cannabis cultivars is used as the basis for synthetic chiral composition formulations of a specific cannabis cultivar, typically after aggregating the data from many samples of the same cannabis cultivar. This raw aggregate data is then refined to remove compounds that are deleterious or otherwise inappropriate to include in the improved synthetic version of the chiral composition. Upon determining the formula for the improved synthetic composition, it is next prepared by adding each element of the formulation via purified feedstocks in the amount specified by each improved synthetic formulation.

FIG. 1 is a flow diagram that illustrates a method 100 of preparing a synthetic chiral composition that possesses organoleptic properties of a plant cultivar according to some embodiments herein. At step 102, the method 100 includes obtaining a sample from the plant cultivar. The sample may be a plant product that includes, but is not limited to, a leaf, a fruit, or a flower isolated from the plant cultivar. The plant cultivar may be from the genus Cannabis, from the family Cannabaceae, from the order Rosales, or from other plants.

At step 104, the method 100 includes analyzing the sample to obtain a stereospecific chemical profile of the sample. The sample may be analyzed by chromatographic techniques. The stereospecific chemical profile of the sample may be obtained via chiral gas chromatography (CGC) or chiral liquid chromatography (CLC). The stereospecific chemical profile may include one or more chemical compounds in the sample and often includes a multitude. In some embodiments, the one or more chemical compounds of the stereospecific chemical profile includes chiral compounds (enantiomers).

At step 106, the method 100 includes analyzing the stereospecific chemical profile of the sample to identify one or more compounds that have organoleptic properties of the plant cultivar. The organoleptic properties may include aroma, flavor or other physiologically detectable characteristics. The one or more compounds in the sample are identified by analytical chemistry techniques such as separating substances from a mixture, compound extraction, chromatography, infrared spectroscopy, nuclear magnetic resonance spectroscopy, or any other analytical chemistry technique. In some embodiments, the one or more identified compounds of the stereospecific chemical profile includes chiral compounds.

At step 108, the method 100 includes quantifying the one or more compounds identified in the sample. The one or more compounds identified in the sample are quantified by mass fraction, percent weight, mole fraction, percentage by volume, or the like. The quantification may be used to determine a ratio of the one or more compounds in the synthetic chiral composition, when preparing the synthetic chiral composition.

At step 110, the method 100 includes preparing the synthetic chiral composition that mimics the organoleptic or physiologically-differentiating properties of the one or more compounds identified in the sample based on the one or more quantified compounds. In some embodiments, the synthetic chiral composition includes either a synthetic or natural form of the one or more compounds identified in the sample in a specific ratio. The synthetic chiral composition may include one or more chiral compounds.

In some embodiments, the synthetic chiral composition includes only synthetic forms of the one or more compounds identified in the sample in a specific ratio. The synthetic chiral composition may include only purified forms of the one or more compounds identified in the sample in a specific ratio. In some embodiments, the synthetic chiral composition includes at least one synthetic form of the one or more compounds identified in the sample in a specific ratio. In some embodiments, the synthetic chiral composition includes at least one purified form of the one or more compounds identified in the sample in a specific ratio. In some embodiments, the synthetic chiral composition includes both the synthetic and purified form of the one or more compounds identified in the sample in a specific ratio. The synthetic form of the one or more compounds may be obtained by chemical or biochemical synthesis. The purified form of the one or more compounds is obtained by at least one of chemical extraction methods and purification techniques to extract and purify the chemical compounds from the plant cultivar. The one or more compounds identified in the sample may include terpene enantiomers, terpenes, terpenoids and a combination thereof.

The synthetic chiral composition may include one or more terpene enantiomers and one or more terpenes or terpenoids. In some embodiments, the one or more terpene enantiomers are pinene isomers, linalool isomers or both. The pinene isomers may be an alpha-pinene enantiomer and a beta-pinene enantiomer. The linalool isomers in the synthetic chiral composition may be a linalool enantiomer. In some embodiments, a ratio of (1R)-(+)-α-pinene to (1S)-(−)-α-pinene in the synthetic chiral composition ranges from 100:1 to 1:100. In some embodiments, a ratio of (1R)-(+)-β-pinene to (1S)-(−)-β-pinene in synthetic chiral composition ranges from 100:1 to 1:100. In some embodiments, a ratio of (S)-(+)-linalool to (R)-(−)-linalool in synthetic chiral composition ranges from 100:1 to 1:100.

In some embodiments, the one or more terpenes or terpenoids in the synthetic chiral composition includes one or more isomers. The one or more isomers may be selected from the group consisting of alpha-bisabolol, borneol, camphene, camphor, delta-3-carene, caryophyllene oxide, alpha-cedrene, beta-eudesmol, fenchol, geraniol, guaiol, alpha-humulene, isoborneol, linalool, menthol, myrcene, nerol, cis-ocimene, trans-ocimene, alpha-phellandrene, alpha-pinene, beta-pinene, sabinene, alpha-terpinene, alpha-terpineol, terpinolene, alpha-guaiene, elemene, farnesene, germacrene, guaia-1(10),11-diene, trans-2-pinanol, selina-3,7(11)-diene, eudesm-7(11)-en-4-ol, and valencene.

In some embodiments, the synthetic chiral composition further includes one or more modifiers selected from the group consisting of thiols, sulfur compounds, simple hydrocarbons, esters, ketones, aldehydes, carboxylic acids, lactones, non-cannabinoid phenols, flavonoids, cannabinoid, and a combination thereof.

In some embodiments, the alpha-pinene enantiomer or enantiomers, the beta-pinene enantiomer or enantiomers, the linalool enantiomer or enantiomers, and the one or more terpenes or terpenoids which may or may not be enantiomerically enriched together include a range of about 2% weight/volume (wt./vol.) to 98% (wt./vol.) of the synthetic chiral composition. In some embodiments, the one or more modifiers included range from about 0.1% weight/volume (wt./vol.) to 90% (wt./vol.) of the synthetic chiral composition.

EXAMPLE 2—Method for generating database comprising synthetic chiral compositions: Each improved synthetic formulation created via the process described in Example 1 and FIG. 1 is then added to a database comprising entries of formulation name, components 1 through N, and amounts for each component 1 through N whereas the total weight % of each component 1 through N is implied by the amounts and totals 100 percent for the entire formulation. In this way, the database comprising synthetic chiral compositions is analogous to a list of recipes.

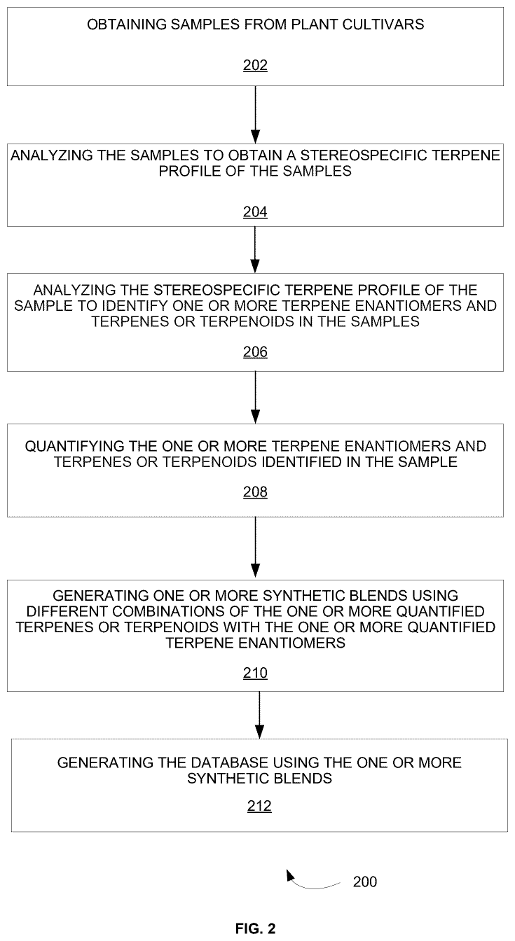

FIG. 2 is a flow diagram that illustrates a method 200 of generating a database of synthetic blends that mimics organoleptic and physiological properties of the light volatile portion of a plant cultivar according to some embodiments herein. At step 202, the method 200 includes obtaining samples from the plant cultivars. The samples may include plant products isolated from the plant cultivars. The plant cultivars may be cannabis varieties or other plants.

At step 204, the method 200 includes analyzing the samples to obtain stereospecific terpene profiles of the samples. The stereospecific terpene profiles of the samples may be obtained by enantiomeric analysis of the samples using a chiral gas chromatography (CGC). Alternatively, or additionally, stereospecific flavonol and flavonoid profiles may be obtained by chiral liquid chromatography (CLC).

At step 206, the method 200 includes analyzing the stereospecific terpene profiles of the samples to identify one or more terpene enantiomers and terpenes or terpenoids in the samples. The one or more terpene enantiomers and terpenes or terpenoids may include organoleptic properties of the plant cultivars. The organoleptic properties may include aroma, flavor or other characteristics.

At step 208, the method 200 includes quantifying the one or more terpene enantiomers and terpenes or terpenoids identified in the samples. The one or more terpene enantiomers and terpenes or terpenoids identified in the samples may be quantified by mass fraction, percent weight, mole fraction, percentage by volume, or the like.

At step 210, the method 200 includes generating one or more synthetic blends using different combinations of the one or more quantified terpenes or terpenoids with the one or more quantified terpene enantiomers. In some embodiments, the one or more quantified terpene enantiomers include an alpha-pinene enantiomer, a beta-pinene enantiomer, a linalool enantiomer or a combination thereof. In some embodiments, the one or more terpenes or terpenoids include alpha-bisabolol, borneol, camphene, camphor, delta-3-carene, caryophyllene oxide, alpha-cedrene, beta-eudesmol, fenchol, geraniol, guaiol, alpha-humulene, isoborneol, linalool, menthol, myrcene, nerol, cis-ocimene, trans-ocimene, alpha-phellandrene, alpha-pinene, beta-pinene, sabinene, alpha-terpinene, alpha-terpineol, terpinolene, alpha-guaiene, elemene, farnesene, germacrene, guaia-1(10),11-diene, trans-2-pinanol, selina-3,7(11)-diene, eudesm-7(11)-en-4-ol, and valencene.

At stepb 212, the method 200 includes generating the database using the one or more synthetic blends generated at the step 210. The database further includes the one or more terpene enantiomers and terpenes or terpenoids identified in the samples with the quantity and ratio information. The data structures included in the database can take multiple forms. Without limitation expressed or implied in the following, one possible data structure is a “peak table” entry consisting of the name or identifier of the molecule that each peak in the chromatogram represents along with the quantity of that molecule in the sample. Another possible data structure involves a “whole chromatogram” entry for each sample that stores an image or coded representation of the chromatogram along with reference information to orient the axes of the chromatogram. These two data structures are the equivalent of storing the information in digital (i.e. peak table) or analog (i.e. whole chromatogram) format. Each data structure has benefits depending on the application by which the database will be utilized. As such, one exemplary example of the database described in step 212 includes both “peak table” AND “whole chromatogram” data structures for each sample.

In some exemplary embodiments, stereo-enriched terpene profiles of cannabis varieties are generated by obtaining 10 separate specimens from 10 different plants for each of 12 different cannabis cultivars and analyzed to identify chiral compounds (i.e. enantiomers). The analytical processes may include methods for preparing the samples, for extracting chemical compounds from the samples, for enantiomeric analysis of the samples and for quantifying the chiral compounds in the samples. The analytical processes may involve chiral gas chromatography or chiral liquid chromatography. The chiral compounds may be quantified by mass fraction, percent weight, mole fraction, percentage by volume. The determined quantities may be used to further determine ratios of chiral compounds to one another in the respective samples. In some embodiments, the determined quantities, ratios, or other chemical properties of the chiral compounds are entered into a database. In some embodiments, the chiral compounds include stereoisomers of terpenes or terpenoids identified in samples.

EXAMPLE 3—Chiral gas chromatographic analyses of cannabis cultivars: Chiral gas chromatographic (CGC) analysis of terpenes was done using a Cyclodex-B column (30 m length, 250 μm internal diameter, 0.25 μm film). Injection was split-less, with the following program: 30° C. for 1 minute, then increase 5° C. min−1 to 100° C., then increase 15° C. min−1 to 250° C., hold for 5 minutes. Chirality was determined by retention index and comparison with authentic stereospecific standards.

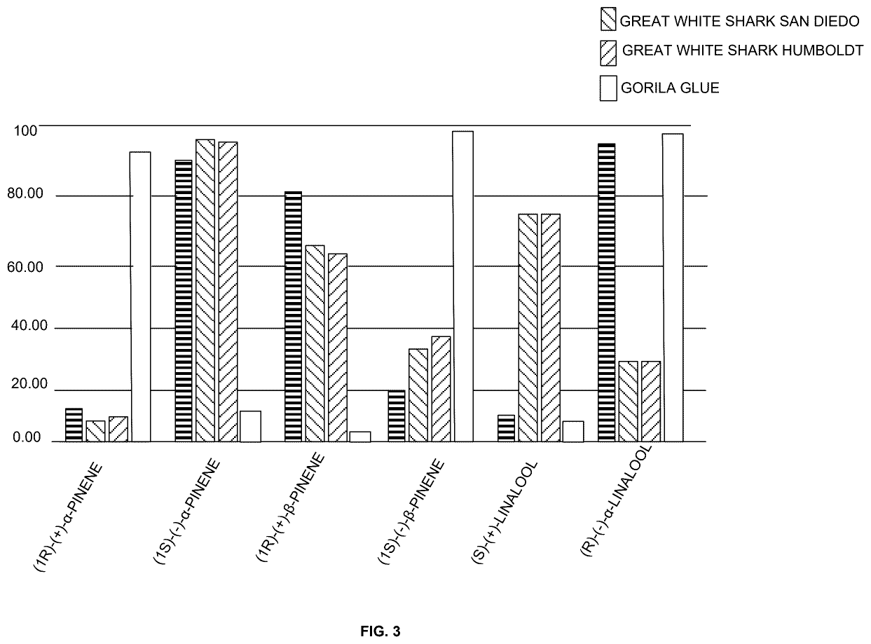

FIG. 3 is a graphical representation that shows results from a series of chiral gas chromatographic analyses of cannabis cultivars indicating the ratios of alpha-pinene, beta-pinene, and linalool enantiomers of some embodiments herein. The data displayed here is in terms of simple enantiomeric ratios and not enantiomeric excess. Stereoisomers of terpenes or terpenoids identified in samples of several different varieties of cannabis have been analyzed and quantified to determine ratios of various terpene enantiomers across different varieties of cannabis. See Table 1 below.

TABLE 1
Ratios of terpene enantiomers across different varieties of cannabis

| | Enantiomeric Ratios of Pinene and Linalool in Cannabis Varieties | | | | | |
|---|---|---|---|---|---|---|
| | pinene | pinene | pinene | pinene | linalool | linalool |
| Cannabis Cultivar | alpha 1 | alpha 2 | beta 1 | beta 2 | 1 | 2 |
| Jack Herer | 9.55 | 90.45 | 80.46 | 19.54 | 4.97 | 95.03 |
| Purple Urkle | 3.68 | 96.32 | 65.64 | 34.36 | 2.46 | 97.54 |
| Sour Diesel | 94.33 | 5.67 | 0.00 | 100.00 | 0.44 | 99.56 |
| Gorilla Glue | 94.84 | 5.16 | 0.29 | 99.71 | 4.18 | 95.82 |
| CBD Remedy | 10.53 | 89.47 | 59.54 | 40.46 | 5.67 | 94.33 |
| Girl Scout Cookies (GSC) | 94.51 | 5.49 | 0.15 | 99.85 | 0.62 | 99.38 |
| Gorilla Glue | 91.49 | 8.51 | 0.21 | 99.79 | 2.68 | 97.32 |
| Green Cush | 24.22 | 75.78 | 34.36 | 65.64 | 2.52 | 97.48 |
| Blue Dream | 4.55 | 95.45 | 76.37 | 23.63 | 4.55 | 95.45 |
| Blue Dream [Santa Cruz] | 4.38 | 95.62 | 76.82 | 23.18 | 4.47 | 95.53 |
| Great White Shark [San Diego] | 2.59 | 97.41 | 64.63 | 35.37 | 75.81 | 24.19 |
| Great White Shark [Humboldt] | 2.69 | 97.31 | 62.17 | 37.83 | 75.32 | 24.68 |
| Bubblegum | 3.39 | 96.61 | 76.31 | 23.69 | 57.10 | 42.90 |
| Sherbert | 84.00 | 16.00 | 0.15 | 99.85 | 0.68 | 99.32 |
| Blueberry | 23.89 | 76.11 | 62.99 | 37.01 | 2.23 | 97.77 |
| XJ-13 | 11.30 | 88.70 | 76.22 | 23.78 | 5.36 | 94.64 |
| Strawberry Cough | 3.89 | 96.11 | 78.94 | 21.06 | 9.55 | 90.45 |
| Gorilla Glue | 88.28 | 11.72 | 0.14 | 99.86 | 2.82 | 97.18 |
| Genius | 87.44 | 12.56 | 0.00 | 100.00 | 0.50 | 99.50 |
| Pot of Gold | 28.53 | 71.47 | 56.61 | 43.39 | 72.73 | 27.27 |
| Golden Pineapple | 17.93 | 82.07 | 69.04 | 30.96 | 3.64 | 96.36 |
| Cotton Candy | 7.79 | 92.21 | 64.89 | 35.11 | 6.03 | 93.97 |

The cultivars of cannabis include Jack Herer, Great White Shark (San Diego), Great White Shark (Humboldt), and Gorilla Glue which are grown under different environmental conditions. In the graphical representation, the identified stereoisomers of terpenes or terpenoids such as pinene isomers and linalool isomers are showed on the X-axis and the quantities of the identified stereoisomers of terpenes or terpenoids are showed on the Y-axis. From the graphical representation, the ratio of the identified stereoisomers of terpenes or terpenoids is determined. From FIG. 3, it is inferred that the ratio of (+)-alpha-pinene to (−)-alpha-pinene; the ratio of (+)-beta-pinene to (−)-beta-pinene and the ratio of (+)-linalool to (−)-linalool are varied from 1:100 to 100:1 across the cannabis strains.

Additional terpenes with stereogenic centers have also been analyzed and can be used to augment those described, including but not limited to, limonene [(−)-limonene and (+)-limonene], alpha-bisabolol [(−)-alpha-bisabolol and (+)-alpha-bisabolol], camphene [(−)-camphene and (+)-camphene], sabinene [(+)-sabinene and (−)-sabinene] 3-carene [(+)-3-carene and (−)-3-carene], and alpha-phellandrene [(+)-alpha-phellandrene and (−)-alpha-phellandrene].

The foregoing description of the specific embodiments will so fully reveal the general nature of the embodiments herein that others can, by applying current knowledge, readily modify and/or adapt for various applications without departing from the generic concept, and, therefore, such adaptations and modifications should be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

We claim:

1. An electronic inhalation device comprising:
a mouthpiece;
a vaporizing element;
a cartridge containing a fluid that is inhalable when vaporized, the fluid comprising:
(a) terpenes including beta-pinene wherein the ratio of (1R)-(+)-β-pinene to (1S)-(−)-β-pinene ranges from about 100:1 to about 1:100 but excluding 1:1;
(b) at least one other terpene or terpenoid; and
(c) cannabinoids comprising at least one of delta-9 THC, delta-8 THC, or CBD, the cannabinoids present in an amount of at least about 20% but no more than about 90% by weight of the liquid composition.

2. The device according to claim 1, wherein the least one other terpene or terpenoid comprises at least one alpha-pinene enantiomer.

3. The device of claim 1, wherein the at least one other terpenes or terpenoids comprises at least one isomer of at least one member selected from the group consisting of alpha-bisabolol, borneol, camphene, camphor, delta-3-carene, caryophyllene oxide, alpha-cedrene, beta-eudesmol, fenchol, geraniol, guaiol, alpha-humulene, isoborneol, menthol, myrcene, nerol, cis-ocimene, trans-ocimene, alpha-phellandrene, sabinene, alpha-terpinene, alpha-terpineol, terpinolene, alpha-guaiene, elemene, farnesene, germacrene, guaia-1(10),11-diene, trans-2-pinanol, selina-3,7(11)-diene, eudesm-7(11)-en-4-ol, and valencene.

4. The device of claim 1, wherein the at least one other terpene or terpenoid consists of at least one enantiomer of limonene, alpha-bisabolol, camphene, sabinene, delta-3-carene, alpha-pinene and/or alpha-phellandrene.

5. The device of claim 1, wherein the composition further comprises at least one modifier selected from the group consisting of thiols, sulfur compounds, simple hydrocarbons, esters, ketones, aldehydes, carboxylic acids, lactones, non-cannabinoid phenols, flavonoids, and a combination thereof.

6. An electronic inhalation device comprising:
a mouthpiece;
a vaporizing element;
a cartridge containing a fluid that is inhalable when vaporized, the fluid comprising:
(a) terpenes including linalool wherein the ratio of (S)-(+)-linalool to (R)-(−)-linalool ranges from about 100:1 to about 1:100 but excluding 1:1;
(b) at least one other terpene or terpenoid; and
(c) cannabinoids comprising at least one of delta-9 THC, delta-8 THC, or CBD, the cannabinoids present in an amount of at least about 20% but no more than about 90% by weight of the liquid composition.

7. The device according to claim 6, wherein the at least one other terpene or terpenoid comprises at least one alpha-pinene enantiomer.

8. The device of claim 6, wherein the at least one other terpene or terpenoid comprises at least one isomer of at least one member selected from the group consisting of alpha-bisabolol, borneol, camphene, camphor, delta-3-carene, caryophyllene oxide, alpha-cedrene, beta-eudesmol, fenchol, geraniol, guaiol, alpha-humulene, isoborneol, menthol, myrcene, nerol, cis-ocimene, trans-ocimene, alpha-phellandrene, sabinene, alpha-terpinene, alpha-terpineol, terpinolene, alpha-guaiene, elemene, farnesene, germacrene, guaia-1(10),11-diene, trans-2-pinanol, selina-3,7(11)-diene, eudesm-7(11)-en-4-ol, and valencene.

9. The device of claim 6, wherein the at least one other terpene or terpenoid consists of at least one enantiomer of limonene, alpha-bisabolol, camphene, sabinene, delta-3-carene, alpha-pinene and/or alpha-phellandrene.

10. The device of claim 6, wherein the composition further comprises at least one modifier selected from the group consisting of thiols, sulfur compounds, simple hydrocarbons, esters, ketones, aldehydes, carboxylic acids, lactones, non-cannabinoid phenols, flavonoids, and a combination thereof.
